(19) United States
(12) Patent Application Publication
(10) Pub. No.: US 2007/0288092 A1
(43) Pub. Date: Dec. 13, 2007

(54) EXPANDABLE INTERVERTEBRAL IMPLANT AND METHOD

(76) Inventor: Nicholas BAMBAKIDIS, Phoenix, AZ (US)

Correspondence Address: DALINA LAW GROUP, P.C., 7910 IVANHOE AVE. #325, LA JOLLA, CA, 92037, US

(21) Appl. No.: 11/670,675

(22) Filed: Feb. 2, 2007

Related U.S. Application Data
(60) Provisional application No. 60/809,810, filed on Jun. 1, 2006.

(51) Int. Cl. A61F 2/44

(52) U.S. Cl. 623/17.11

(57) ABSTRACT

Replaces anterior spinal column with an expandable lattice implant made of supportive material that includes use of fusion augmenting material. Allows ease of use while promoting both immediate spinal stability and eventual arthrodesis. May be utilized during anterior or retroperitoneal approaches to the spinal column, primarily addressing anterior spinal column pathology. May utilize fusion augmenting material of limited biomechanical strength compared to the strength of its rigid components. May be accompanied by additional anterior or posterior spinal instrumentation and fixation. Uses a pair of circular endplate discs that are distracted from each other by ribs of rigid support rods. Extension may be performed using an expansion tool once device is placed within intervertebral space. Hollow portions of device may be packed with bone or other materials to enhance eventual fusion. Shape of discs at each end may be manipulated prior to surgery to adapt to the specific spinal curvature desired.

This application claims benefit of U.S. Provisional Patent Application Ser. No. 60/809,810 filed Jun. 1, 2006, the specification of which is hereby incorporated herein by reference.

### BACKGROUND OF THE INVENTION

1. Field of the Invention

Embodiments of the invention described herein pertain to the field of medical devices and methods. More particularly, but not by way of limitation, one or more embodiments of the invention enable an expandable intervertebral implant and method.

2. Description of the Related Art

The ideal implant is one that restores the normal alignment of the spinal column and provides biomechanical stability. This is provided in the short term by immediate spinal fixation utilizing metallic implants such as titanium or stainless steel. The difficulty with such implants in the long term is that a lack of solid bony fusion (pseudoarthrosis) may lead eventually to hardware failure and subsidence of the implant. The utilization of bone only as an implant is complicated by its difficulty in accurately sizing the graft and subsidence of the material. Because of the sometimes narrow corridors through which surgical access to the implant site is afforded, the utilization of expandable interbody devices has intuitive advantages. Excessive machining of bone-only implants is avoided as well, and a single implant size may be utilized for many different applications. Partial correction of the deformity may also be achieved.

Prior patents have described expandable intervertebral devices utilizing a variety of different mechanisms and proposing numerous different advantages (U.S. Pat. No. 5,665,122, No. 5,505,732, No. 5,653,762, No. 6,183,517, No. 6,193,757, No. 7,044,971, No. 5,059,193, No. 6,102,950, No. 6,126,689, No. 5,749,916). These devices, and others described in published patent applications, are limited in many ways. Most are designed for use in situations arising solely from degenerative conditions of the intervertebral disc. They are thus not designed to expand beyond a relatively small size and cannot replace a vertebral body following a corpectomy. Devices made from titanium or other metallic substances may not allow for eventual bony fusion even though they provide immediate stability and may be prone to eventual pseudoarthrosis and implant subsidence. This may be true even if the device is designed to be hollow to allow for the placement of bone graft material, because only large openings and large contact surfaces at the endplates above and below the implant will allow for eventual solid bony fusion (arthrodesis).

For at least the limitations described above there is a need for an expandable intervertebral implant and method.

### BRIEF SUMMARY OF THE INVENTION

Embodiments of the invention are designed to replace disc material or bone utilized during surgical stabilization of the spine. Specifically, embodiments of the invention incorporate a rigid external frame designed to expand to fit the desired space along with additional fusion augmenting material leading to the enhancement of arthrodesis.

The combination of intervetebral disc material and the solid bone of the vertebral body together form a stable support structure for the mammalian spine. In humans, several types of disease processes may result in partial or complete destruction of these support structures. These may include but are not limited to arthritic degeneration, infection, traumatic disruption, or neoplasm. During the course of treating these conditions surgically it is sometimes necessary to remove all or part of both the discs (discectomies) and/or the vertebral bodies (corpectomies). This results in biomechanical destabilization of the spine necessitating replacement with either bone or an implanted device.

Embodiments of the implant are designed to replace the anterior spinal column by an expandable lattice implant made of solid supportive material. This cage can be sheathed with fusion material both around its cylindrical shaft and at its ends. This allows for maximal ease of use in a variety of clinical applications while promoting both immediate spinal stability and eventual arthrodesis. Embodiments may be utilized during anterior or retroperitoneal approaches to the spinal column, primarily addressing anterior spinal column pathology. Since the device may include incorporation of fusion augmenting material of limited biomechanical strength when compared to the strength of its rigid components, it may be accompanied by additional anterior or posterior spinal instrumentation and fixation. This is normally performed in cases of anterior intervertebral implant placement in such applications and is not a drawback to this device design.

To achieve these objectives, a pair of circular endplate discs are distracted from each other by ribs of rigid support rods. This may be performed using an expansion tool once the device is placed within the intervertebral space. The hollow portions of the device may be packed with bone or other materials to enhance eventual fusion. The shape of the discs at each end may be manipulated prior to manufacture so that a variety of shapes are available to the operating surgeon thus maintaining adaptability to the specific spinal curvature desired. The use of fusion augmenting material in the interstices of the metallic lattice provides additional biomechanical strength following initial implantation while maximizing the probability of eventual arthrodesis.

### BRIEF DESCRIPTION OF THE DRAWINGS

The above and other aspects, features and advantages of the invention will be more apparent from the following more particular description thereof, presented in conjunction with the following drawings wherein:

FIG. 1 is an end on view of an embodiment of the device.

FIG. 2 is a side view of an embodiment of the device comprising the saucer like endplate engagers and the intervening cylindrical metallic-bioabsorbable lattice.

FIG. 3 is a side of an embodiment of the device that incorporates views in the compressed (a) and expanded (b) states.

FIG. 4 is an exploded view of an embodiment of the expandable design elements of the cylindrical shaft lattice.

FIG. 5 is a view from the side of an embodiment of the device demonstrating the use of the insertion tool in expanding the device.

FIG. 6 is an end view of an embodiment of the device showing the placement of the insertion tool.

FIG. 7 illustrates a flow chart for manufacturing an expandable intervertebral fusion implant in accordance with at least one embodiment of the invention.

### DETAILED DESCRIPTION

An expandable intervertebral implant and method will now be described. In the following exemplary description numerous specific details are set forth in order to provide a more thorough understanding of embodiments of the invention. It will be apparent, however, to an artisan of ordinary skill that the present invention may be practiced without incorporating all aspects of the specific details described herein. In other instances, specific features, quantities, or measurements well known to those of ordinary skill in the art have not been described in detail so as not to obscure the invention. Readers should note that although examples of the invention are set forth herein, the claims, and the full scope of any equivalents, are what define the metes and bounds of the invention.

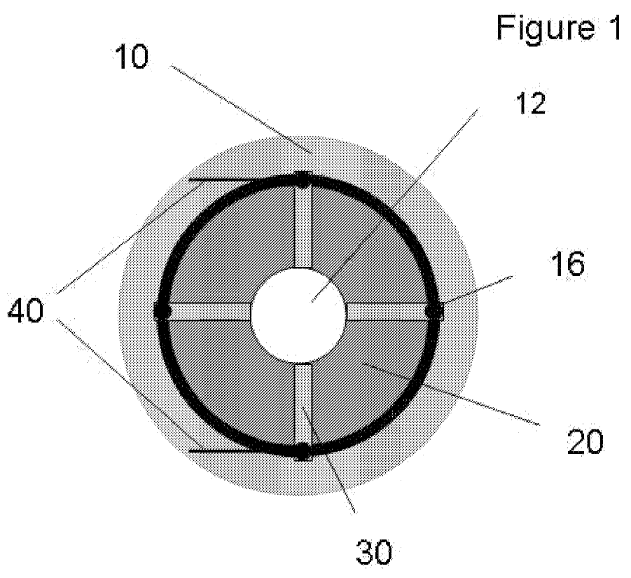

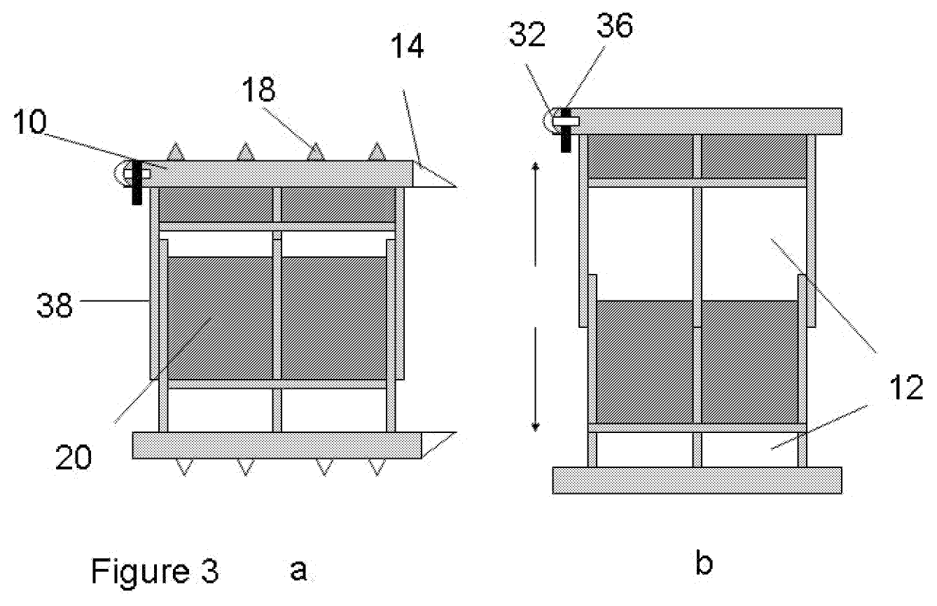

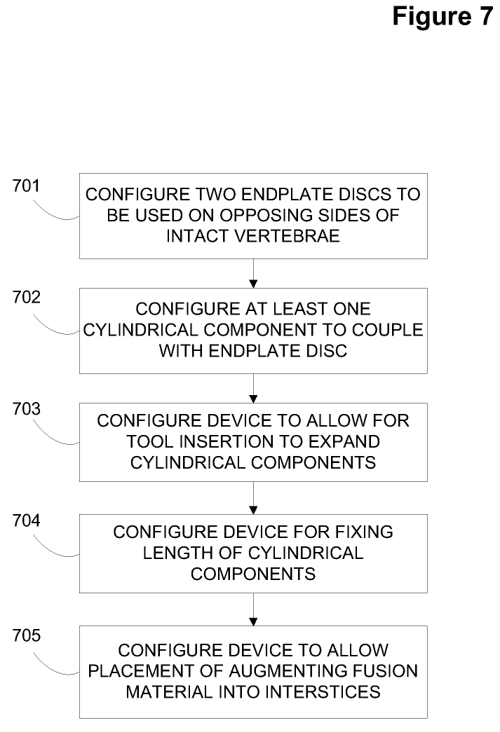

An embodiment of the expandable intervertebral fusion implant appears in FIGS. 1-6. A flowchart for manufacturing an embodiment of the device appears in FIG. 7. FIG. 1 is an end on view of an embodiment of the device. An embodiment of the implant includes endplate discs 10 from which protrude perpendicular struts 30. Protruding from the vertebral body-contacting surface of the endplate discs are multiple teeth or ridges 18 (see FIG. 3) which prevent migration of the implant by keeping teeth/ridges firmly embedded within the vertebral body.

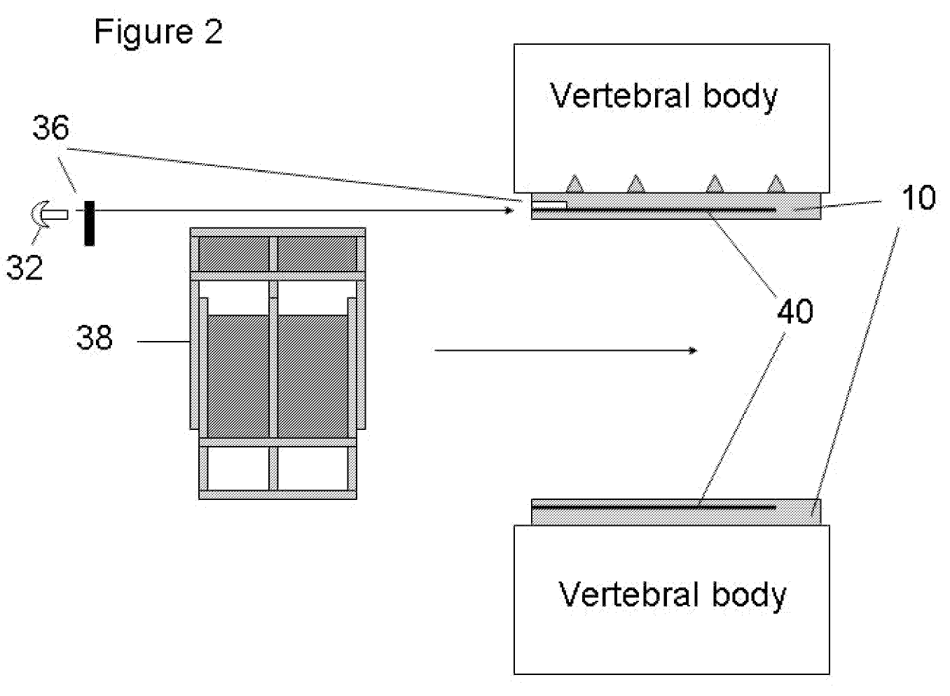

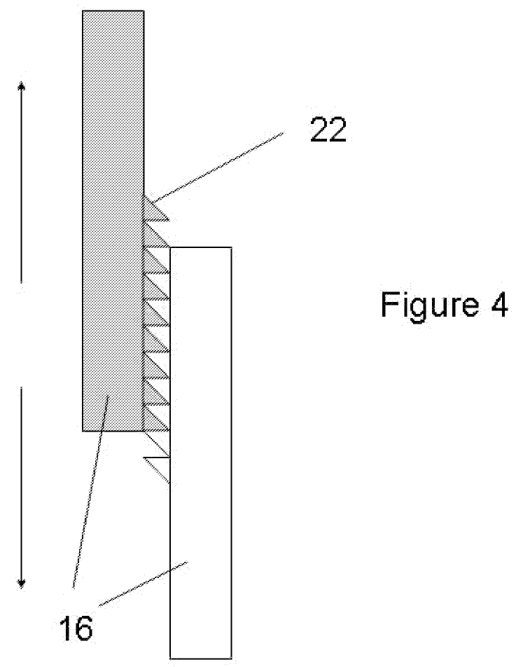

FIG. 2 is a side view of an embodiment of the device comprising the saucer like endplate engagers and the intervening cylindrical metallic-bioabsorbable lattice. The body of the implant, or cylindrical component 38, includes a lattice of rigid support rods 16 (pointing into the page as shown in FIG. 1 or shown as a side view in FIG. 4) and is designed as a separate structure for insertion into a space between adjacent vertebral bodies in the spine. Two or more of cylindrical component 38 may be utilized to form the body when at least one cylindrical component 38 is sized to fit within another cylindrical component. This allows for extension of the body. A variety of sizes of the cylindrical component 38 may be made available depending on the application following but not limited to discectomy or corpectomy. Fusion augmenting material 20 (see FIGS. 1-3,5) may make up large volumes between the rigid structures of the device and is denoted as dark grey shading. The body of the device is configured to securely hold fusion augmenting material and any means or method of holding the fusion augmenting material within the device is in keeping with the spirit of the invention.

As used herein, the term fusion augmenting material defines one or more bioabsorbable, porous, and/or graft materials that promote the growth of bone tissue from one vertebral body across a disc space to an adjacent vertebral body to thereby substantially eliminate relative motion between those vertebrae. The graft material refers to bone materials, autologous or any other material that may be utilized to graft, including cancellous bone for example.

A typical endplate disc 10 is shaped to allow the placement against the vertebral body around the circumference and has a hollow center to allow for the placement of the fusion augmenting material 20 to contact the vertebral body. The perpendicular struts 30 acts as load sharing structures and may provide scaffolds for attaching the fusion augmenting material 20. Any other arrangement that provides a manner of placement of endplate discs onto vertebral body and provide hardware to couple and secure the cylindrical component 38 is keeping the spirit of the invention. For example, an endplate discs may be configured with swing-arms to wrap around the vertebral body or may be a pair of two U-shaped pieces that interlock with each other after assembly on the vertebral body.

Open spaces 12 are provided through which additional fusion material may be placed into the interstices of the device. (See also FIG. 3) The placement of the additional fusion material may be performed after the assembly and expansion of the cylindrical component 38. This is also to encourage bone growth through the center of the device leading to a solid fusion. The additional fusion material may include autologous bone material, such as cancellous bone for example.

The rigid portions of the device (light grey) such as endplate disc 10 and perpendicular struts 30 for example may be made of biocompatible materials such as stainless steel, titanium, graphite, ceramic, or various plastics or composites. The portions promoting bony fusion (dark grey) may be made of a fusion augmenting material through which bony fusion may occur. The exact material chosen depends on the specific application.

FIG. 2 shows the manner in which the two main components of the device are designed to fit together from a side perspective. Stylized models of the vertebral bodies above and below the implant are shown, and removal of the intervening anatomic structures has taken place. The endplate discs 10 have been placed against the vertebral bodies. Additional modification to the curvature, thickness, or shape of the endplate discs 14 may be made in order to alter the degree of lordotic or kyphotic angle desired. This may be done at the time of manufacture and a variety of sizes and shapes may then be made available at the time of implantation. Placement of the cylindrical component 38 may be performed along sunken rails 40 on the endplate discs 10 which have been machined to fit the radius and depth of piece 38 precisely. This may be done either before or after device implantation. The surgical approach may be done through an anterior or retroperitoneal approach, and the endplates of the vertebral bodies above and below the device may be prepared to promote bone fusion.

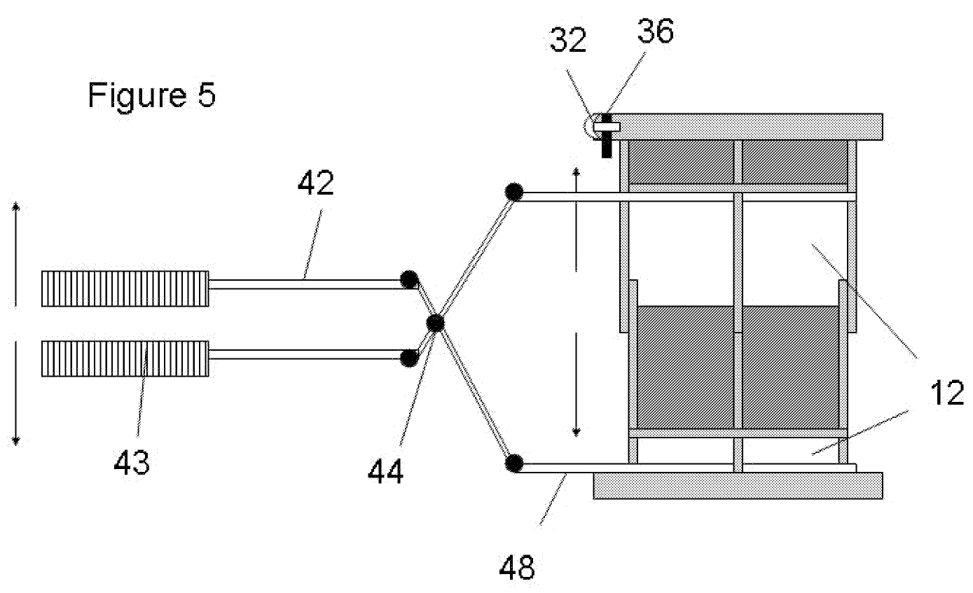

FIG. 3 is a side of an embodiment of the device that incorporates views in the compressed (a) and expanded (b) states. Expansion of the device occurs along the long axis of the cylindrical component using an external expansion tool. Following adequate distraction and expansion, the locking plate 36 is placed in a groove along the cylindrical component 38 and into the endplate disc 10. This assembly is secured by screw 32 which must be designed to be self-locking in order to prevent inadvertent loosening. (See also FIG. 2 for exploded view with locking plate 36 and screw 32 shown to the left of cylindrical component 38 in anticipation of insertion to the right to couple and lock cylindrical component 38 to endplate disc 10). The screw and locking plate are not shown on the bottom endplate disc in FIGS. 3 and 5 are optional if a biomechanical test indicates that the cylindrical component can be secured by coupling through the sunken rails of the endplate discs without additional locking. Additional expansion of the implant may be carried out at any time following assembly. Assembly may also precede implantation, so that sunken rails 40 of the endplate discs 10 are substantially parallel to allow coupling of the cylindrical component 38 to the endplate discs 10.

FIG. 4 shows a magnified view of the interface between the support rods 16 running along the long axis of cylindrical component 38. Interdigitating teeth 22 are integrated in the rods and oriented so that expansion in the direction of the arrows may occur but compression may not. Such teeth may be utilized or arranged along all potential mating surfaces of the longitudinal rods at each of the four corners of the device as shown in FIG. 1. Any number of support rods or any other arrangement that provides a manner of providing longitudinal expansion between the endplate discs 10 is in keeping with the spirit of the invention.

Removal of the device may be performed after removal of screw 32 and locking plate 36 as per FIG. 3. Because the rails 40 are designed to fit the cylindrical component 3, this portion may be then removed using a removal tool without compression of the device. The endplate discs may then be pried from their positions and removed. Any other arrangement that provides a manner of securing and the subsequent removal of cylindrical component 38 to/from endplate discs 10 is in keeping with the spirit of the invention.

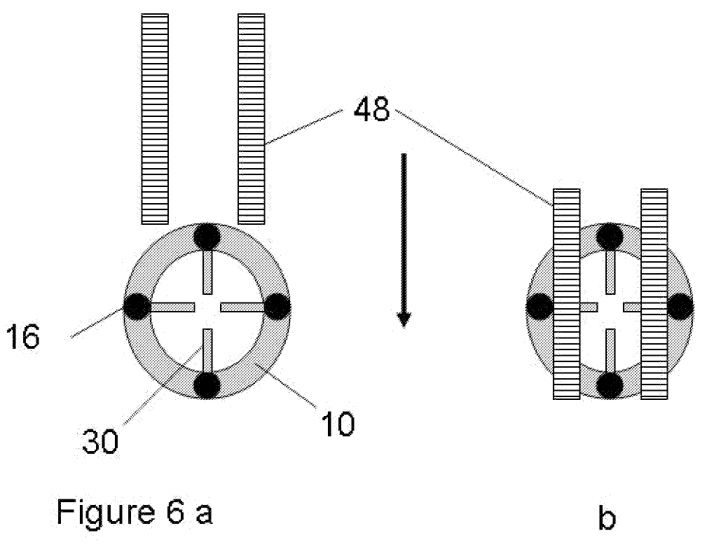

Further elaboration of the process of expansion for the device is shown in FIG. 5. From a side view, the expansion tool 42 is handled at ends 43 and slid forward to engage the circular supports 10 (also see FIG. 3). This may be done either at the endplates or at the mid-position of the cylindrical component. The position of the footplates 48 of the expansion tool are shown from a lateral view in FIG. 5 as well as in an end on view in FIG. 6. This is shown both before (a) and after (b) insertion. The footplates 48 are placed medial to the support rods 16 and above the perpendicular struts 30. After engagement of the tool in the device, expansion is performed by moving the grips 43 away from each other around the pivot points 44.

FIG. 7 illustrates a flow chart for manufacturing an expandable intervertebral fusion implant in accordance with at least one embodiment of the invention. Embodiments of the invention may be utilized after preparing the spinal column and removing damaged vertebral materials, including but not limited to damaged disc nucleus, between two intact vertebrae. However, at any time preceding use of the device, the device may be manufactured according to FIG. 7.

The process of manufacturing an embodiment of the invention starts at 701 by configuring two endplate discs so that they may be utilized on opposing sides of two adjacent intact vertebrae. This for example may include the addition of teeth to the endplate discs so that they are configured to remain stationary when placed against the vertebrae. The cylindrical component is then configured to be coupled to the two embedded endplate discs at 702. The step for example may include the machining of lips on the ends of the cylindrical components and/or rails into the endplate discs so that a cylindrical body may be inserted into the discs at a later time. The device is configured to allow for an expansion tool to be inserted into the device at 703. For example, machining of spaces or leaving gaps in the device that allow for the insertion of the tool are in keeping with the spirit of this step. The expansion tool is used to expand the cylindrical component so that the endplate discs bears against the vertebral bodies after time of manufacture. At time of manufacture however, the expansion tool may be inserted to test for proper expansion of the device before time of surgery. The device is configured for fixing the length of the cylindrical component at 704. This may include for example the inclusion of interdigitating teeth 22 on support rods 16 or any other mechanism which allows the device to be expanded to a fixed length. The device is configured to allow the placement of additional fusion material into embodiments of the device at 705. This may include for example the addition of struts or spaces or gaps that allow for any type of fusion augmenting material to be added at the time of manufacture or at the time of surgery.

While the invention herein disclosed has been described by means of specific embodiments and applications thereof, numerous modifications and variations could be made thereto by those skilled in the art without departing from the scope of the invention set forth in the claims.

What is claimed is:

1. An expandable intervertebral implant comprising:
a pair of endplate discs comprising a vertebral body contacting side and a cylindrical component contacting side;
a first and second cylindrical component configured to couple with one another wherein said first and said second cylindrical component when coupled with one another allow for an expanded position and a collapsed position;
a plurality of support rods coupled to said first and said second cylindrical component;
at least one strut configured to hold fusion augmenting material within said first and said second cylindrical component; and,
said cylindrical component configured to couple with said pair of endplate discs and configured to expand toward opposing vertebral bodies.

2. The expandable intervertebral implant of the claim 1, wherein endplate disc comprises a plurality of teeth on said vertebral body contacting side.

3. The expandable intervertebral implant of the claim 1, wherein support rods comprise interdigitating teeth configured to allow for extension of said support rods.

4. The expandable intervertebral implant of the claim 1, wherein said expandable intervertebral implant is formed of one or more biocompatible materials.

5. The expandable intervertebral implant of claim 4, wherein said pair of endplate discs and said first and said second cylindrical component are formed of one or more rigid biocompatible materials selected from the group consisting of stainless steel, titanium, graphite, ceramic, plastics, and composites.

6. The expandable intervertebral implant of the claim 1, wherein said expandable intervertebral implant comprises interstices configured for placement of fusion augmenting material.

7. The expandable intervertebral implant of the claim 5, wherein said fusion augmenting material comprises a bone material.

8. The expandable intervertebral implant of the claim 1, wherein said intervertebral implant is configured for engagement with an external expansion tool.

9. The expandable intervertebral implant of the claim 1, wherein said pair of endplate discs are configured to provide lordotic or kyphotic curvature to a spinal column.

10. A method for manufacturing an expandable intervertebral implant comprising: configuring a pair of endplate discs with vertebral body contacting sides for contact with opposing sides of intact vertebrae;
configuring a first and second cylindrical component to couple with one another wherein said first cylindrical component configured to fit inside said second cylindrical component wherein said first and said second cylindrical component are configured to couple with said pair of endplate discs;
configuring said first and said second cylindrical component to allow for insertion of an expansion tool;
configuring said first and said second cylindrical component to allow for a set expanded length; and,
configuring said first and said second cylindrical component to allow for insertion of fusion augmenting material into interstices of said first and said second cylindrical component.

11. An expandable intervertebral implant comprising:
means for configuring a pair of endplate discs with vertebral body contacting sides for contact with opposing sides of intact vertebrae;
means for configuring a pair of endplate discs with vertebral body contacting sides for contact with opposing sides of intact vertebrae;
means for configuring a first and second cylindrical component to couple with one another wherein said first cylindrical component configured to fit inside said second cylindrical component wherein said first and said second cylindrical component are configured to couple with said pair of endplate discs;
means for configuring said first and said second cylindrical component to allow for insertion of an expansion tool;
means for configuring said first and said second cylindrical component to allow for a set expanded length; and,
means for configuring said first and said second cylindrical component to allow for insertion of fusion augmenting material into interstices of said first and said second cylindrical component.
